(19) United States
(12) Patent Application Publication
(10) Pub. No.: US 2004/0116966 A1
(43) Pub. Date: Jun. 17, 2004

(54) Methods for implementing a public access defibrillation (PAD) program

(75) Inventor: Jeffrey P. Calibaba, Calgary (CA)

Correspondence Address: Michael C. Barrett, Esq., FULBRIGHT & JAWORSKI, L.L.P., Suite 2400, 600 Congress Avenue, Austin, TX, 78701, US

(73) Assignee: Access Medical Inc.

(21) Appl. No.: 10/675,861

(22) Filed: Sep. 30, 2003

Related U.S. Application Data
(60) Provisional application No. 60/415,219, filed on Sep. 30, 2002.

(51) Int. Cl. A61N 1/39

(52) U.S. Cl. 607/5

(57) ABSTRACT

Methods for implementing a public access defibrillation (PAD) program. In a representative embodiment, the following steps are done: identifying a domain for the program; developing a strategic plan for the program; conducting initial activities for setting up the program; presenting details for implementing the program within the domain; launching the program in the domain; publicly proclaiming the domain a PAD community or organization if launching is successful; and mapping the domain as unsuccessful if launch is not successful.

This application claims priority to pending U.S. provisional application serial No. 60/415,219, which was filed Sep. 30, 2002. That entire application, including all of its appendices, is incorporated here by reference.

### FIELD OF THE INVENTION

The present invention generally relates to Public Access Defibrillation (PAD), and more particularly relates to methods for implementing a PAD program.

### BACKGROUND OF THE INVENTION

Sudden Cardiac Arrest (SCA) is a condition in which the heart stops beating suddenly and unexpectedly due to a malfunction in the electrical system of the heart. The malfunction that causes SCA is a life-threatening abnormal rhythm, or arrhythmia. The most common arrhythmia is Ventricular Fibrillation (VF). When in VF, the heart's rhythm is so chaotic that the heart merely quivers, and is unable to pump blood to the body and brain. This chaotic rhythm is generally referred to as fibrillation.

Unfortunately, anyone can suffer SCA. SCA is unpredictable and it can happen at anytime and any place. As illustrated in FIG. 1, it is estimated that approximately three hundred thousand (300,000) deaths per year are attributable to SCA. This number of deaths attributable to SCA is greater than the number of deaths attributed to Acquired Immune Deficiency syndrome (AIDS), Breast Cancer, Lung Cancer, or Stroke.

A victim in SCA first loses his or her pulse, then consciousness, and finally the ability to breath. These events happen in a matter of seconds. An effective treatment for SCA is to deliver an electrical shock using a device called a defibrillator (i.e., to defibrillate the heart). Voltage stored by the defibrillator pushes electrical current through the heart by means of electrodes or paddles place on the victim's body, preferably the victim's chest. The brief pulse of current halts the heart fibrillation, giving it a chance to start beating with a normal rhythm. This delivering of the electrical shock, which returns the heart to normal rhythm, is called defibrillation.

Survival rates for SCA are the highest when defibrillation is conducted within the first few minutes of an arrest, and the person has the best chance of survival if the defibrillation shock is given within the first three minutes of the person's collapse. As illustrated in FIG. 2, one study has shown that the chances of resuscitating an individual suffering SCA are reduced by about seven percent (7%) to about ten percent (10%) with each minute that lapses between the SCA and application of the defibrillation shock. Therefore, rate of survival for SCA victims average less than two percent (2%) when defibrillation is delayed ten (10) minutes or more. Furthermore, if normal heart rhythm is not obtained within the first four (4) to six (6) minutes after the arrest, the victim may sustain irreversible brain damage.

One medical device that has been developed to reduce the time that lapses between the SCA and defibrillation is an Automated External Defibrillator (AED). Generally, an AED is a small, portable device that analyzes the heart's rhythm and prompts a user to deliver a defibrillation shock if it determines the need for such a shock. Once the AED is activated, it guides the user through each step of the defibrillation process by providing voice and/or visual prompts.

AEDs are specially designed for use by a “first responder,” who would be the first person to typically arrive on the scene of a medical emergency. A first responder can be an emergency medical services worker, a firefighter or a police officer, or it can be a layperson with minimal or no AED training. Time to defibrillation can be reduced if an AED is “on-site” and can be quickly brought to the victim. This is one of the reasons that survival rates are significantly improved in communities/organizations with PAD programs.

PAD programs provide strategic and wider deployment of AEDs in private, public, or governmental venues, or any other venue, for trained, minimally trained or even untrained personnel. With strategic and wider deployment of AEDs, the AEDs are placed in the hands of people, and in key locations where people congregate: wherever people live, work, study, and/or play, which enables the person on-scene, even someone with minimal or no training, to give a lifesaving shock.

Accordingly, it is desirable to provide methods for implementing a PAD program. Furthermore, other desirable features and characteristics of the present invention will become apparent from the subsequent detailed description of the invention, taken in conjunction with this background of the invention, drawings, and appendices.

### BRIEF DESCRIPTION OF THE APPENDICES

The present invention will hereinafter be described in conjunction with the following appendices (filed with U.S. provisional patent application serial No. 60/415,219 and incorporated by reference here):

APPENDIX A is “Getting Started”;

APPENDIX B is “Communicating Your Message”;

APPENDIX C is the “Tool Box”;

APPENDIX D is an overview of the method for implementing a PAD program;

APPENDIX E is a second overview of the method for implementing a PAD program; and

APPENDIX F is strategic planning workshop exercises.

### BRIEF DESCRIPTION OF THE DRAWINGS

The present invention will hereinafter be described in conjunction with the following illustrations or drawings, and the illustrations or drawings provided in the appendices, and any other document incorporated therein by reference:

FIG. 1 is a graph illustrating the estimated number of death per year that are attributable to SCA, AIDS, breast cancer, lung cancer, and stroke;

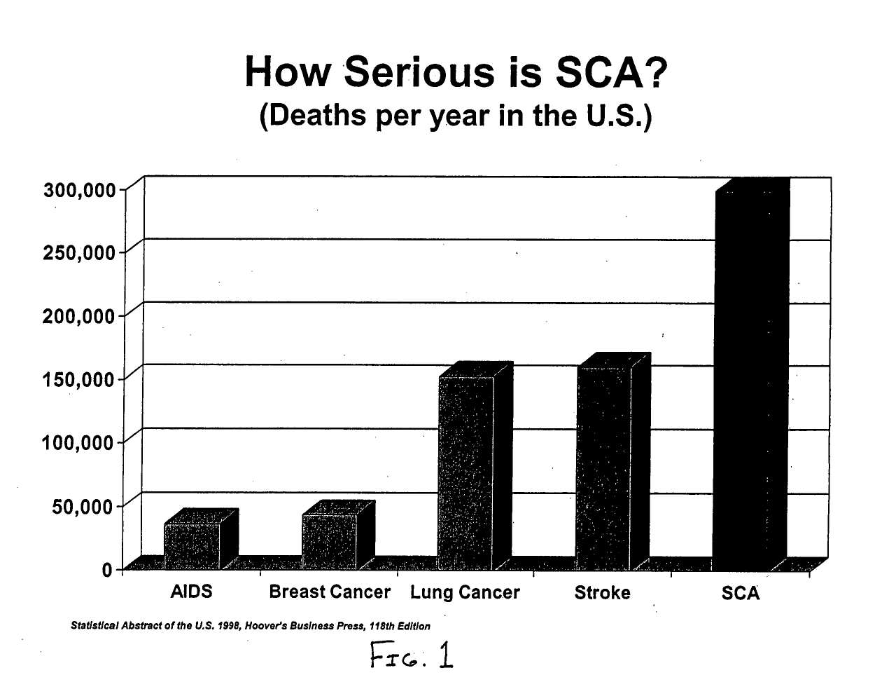

FIG. 2 is a graph illustrating the relationship between resuscitation success and time;

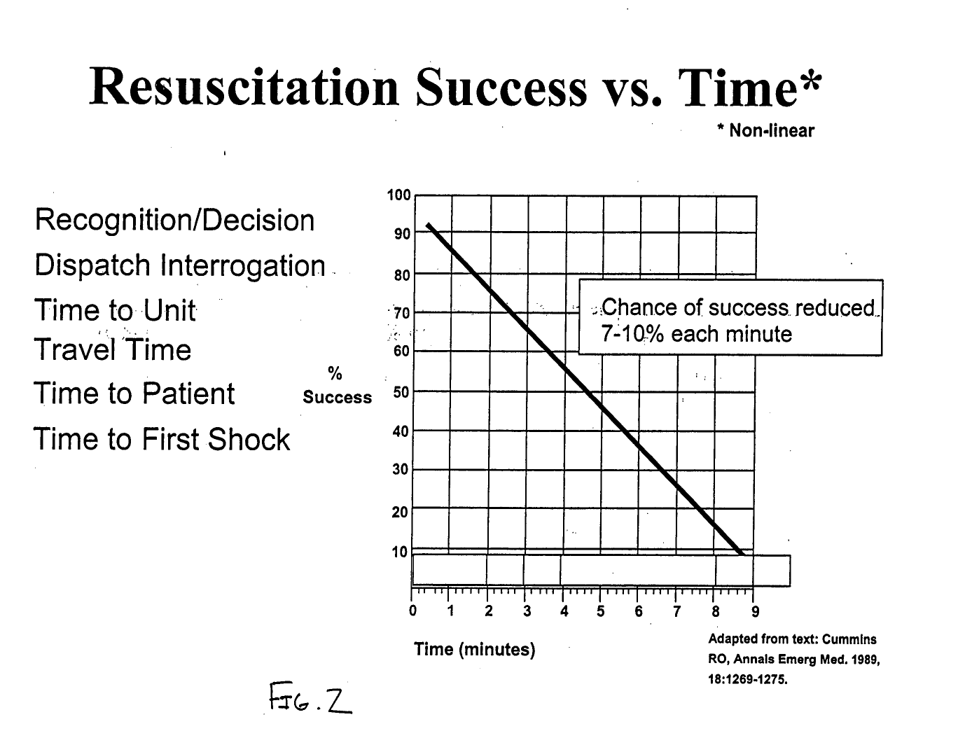

FIG. 3 is a flowchart illustrating steps associated with the PAD program in accordance with a first embodiment of the present invention;

FIG. 4 is a flowchart of the steps that can comprise the developing a strategic plan for a PAD;

FIG. 5 is a flowchart of the steps that can comprise the conducting initial activities for setting up the PAD program of FIG. 3;

FIG. 6 is an illustration of the steps associated with the PAD program in accordance with a second embodiment of the present invention;

FIG. 7 is a flowchart illustrating the steps associated with the PAD program in accordance with a third embodiment of the present invention; and

FIG. 8 is an exemplary illustration of private, public, and governmental that can utilize the HeartSave Awareness™ program in accordance with an embodiment of the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention, the following detailed description of the invention, which includes the appendices.

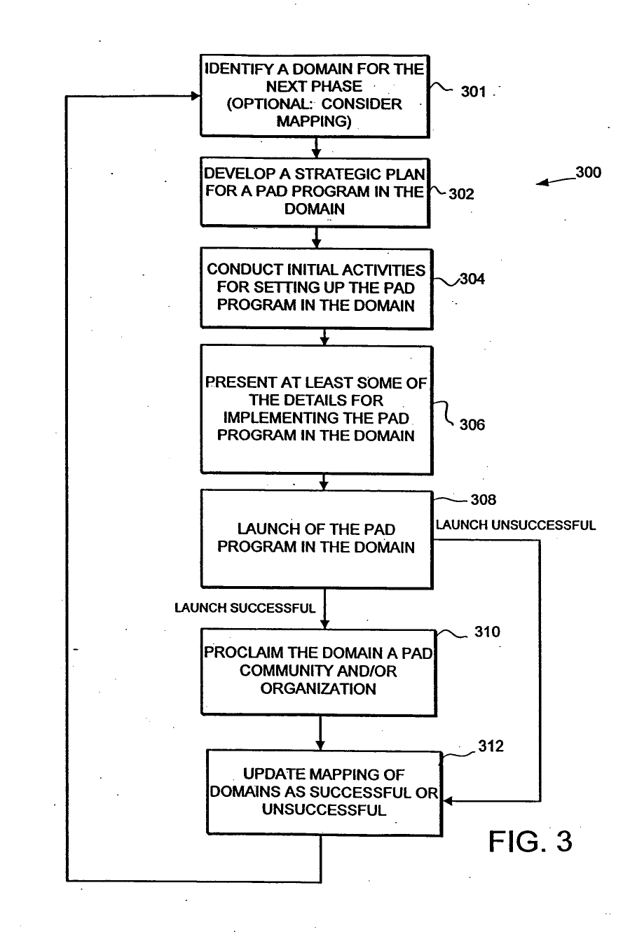

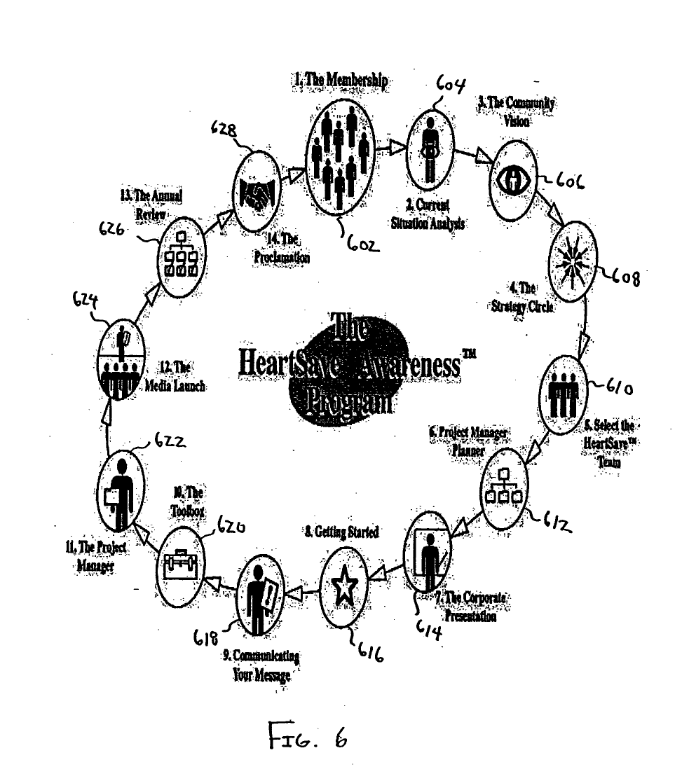

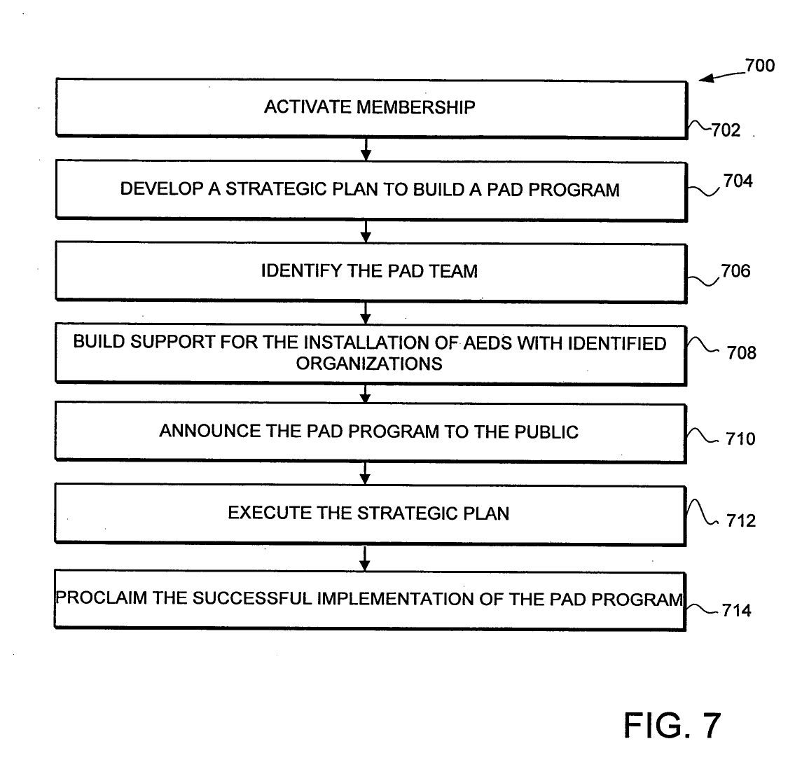

Generally, the methods of the present invention provide for implementation of a PAD program, which will also be referred to herein as the “HeartSave Awareness™ program.” The methods or the present invention can utilize any number of the steps or any combination of a subset of the steps subsequently described in this detailed description of the invention or the appendices. For example, in accordance with a first embodiment of the present invention, the method for implementing a PAD program comprises seven (7) steps as subsequently described in this detailed description of the invention and illustrated with reference to FIG. 3. In accordance with a second embodiment of the present invention, the PAD program (i.e., the HeartSave Awareness™ program) is a method with fourteen (14) steps (i.e., steps 602 through 628) as illustrated in FIG. 6, and described in the appendices. In accordance with a third embodiment of the present invention, the HeartSave Awareness™ program is a method 500 with seven (7) steps (702,704,706,708,710,712,714) as illustrated in FIG. 7. Furthermore, while the steps are presented in this detailed description of the invention in an exemplary order of execution, the steps can be conducted in any sequential order, serially, or in parallel according to the present invention, and any of the steps of the three methods are applicable to all three methods and it should be understood that some of the steps are optional.

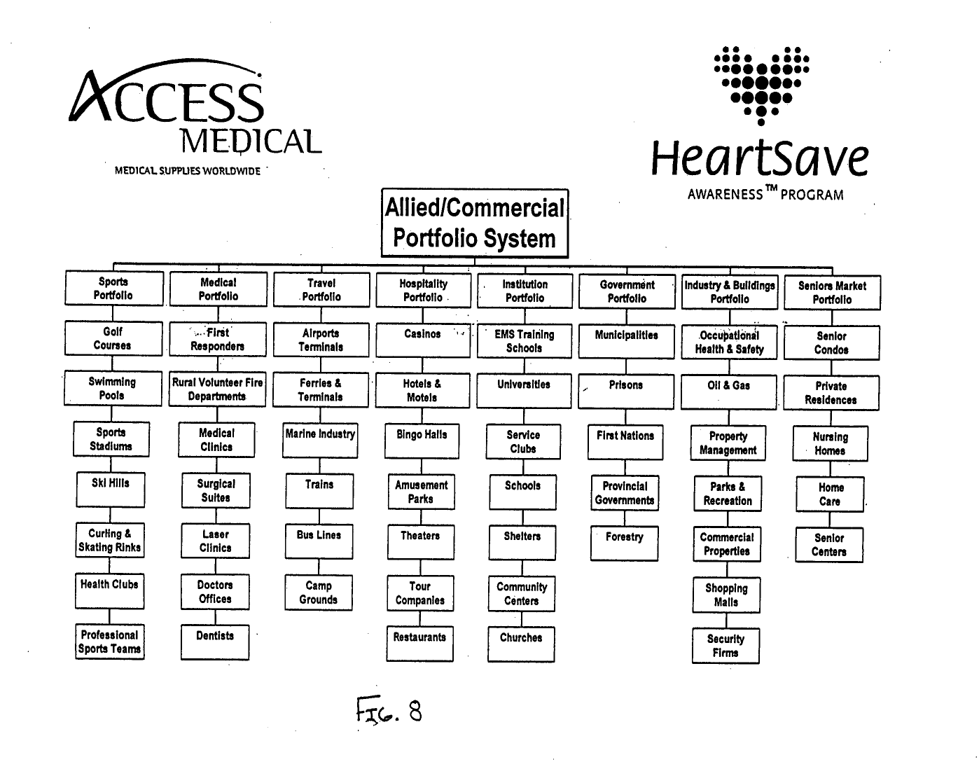

The HeartSave Awareness™ program empowers emergency services, communities, and other organizations to focus on a well defined and comprehensive plan to address the vital health care issue of SCA. This program preferably involves several layers of laypersons, paraprofessionals and professionals, each with diverse competency and skill set levels. The relationships of the individuals and the processes come together in a plan to increase SCA survival rates. The HeartSave Awareness™ program can be developed for any number of domains, which are private, public, governmental organizations and/or communities, or a combination of these organizations and/or communities. For example, the HeartSave Awareness™ program can be developed for one or more sporting, medical, travel, hospitality, institutional, governmental, industry, and/or senior organizations as illustrated in FIG. 8.

Referring to FIG. 3, the method for implementing a PAD program (i.e., a HeartSave Awareness™ program) 300 is illustrated in accordance with a first embodiment of the present invention. Additional details of this method 300 can be found in the attached appendices, and it should be understood that the some of these steps are optional. The method 300 preferably begins with identifying a domain for the next phase 301 and optionally mapping.

After identifying the domain for the next phase 301 and optionally mapping, the method 300 continues with developing a strategic plan for the PAD in the domain 302. Developing the strategic plan for the PAD in the domain 302 is preferably conducted with members from Emergency Services, such as senior management and public/media relations personnel. This internal group of Emergency Services, with its understanding of the dynamics of their organization and/or community, is well suited to assist in developing the strategic plan for the PAD in their organization and/or community. However, developing the strategic plan for the PAD in the domain 302 can be conducted without members from Emergency Services in accordance with the present invention.

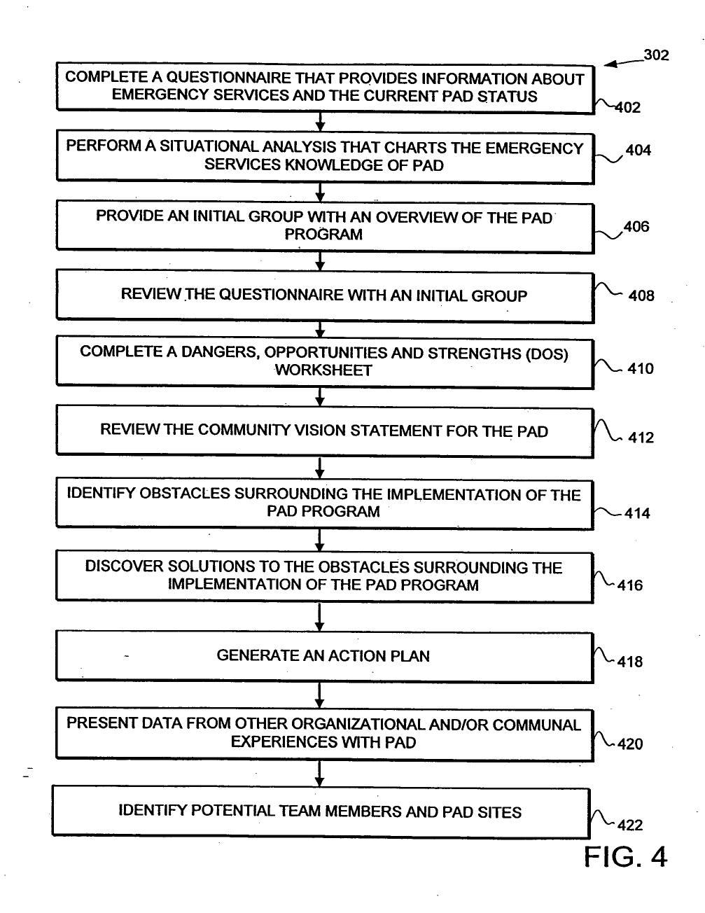

Referring to FIG. 4, developing a strategic plan for the PAD in the domain 302 with members from Emergency Services is beneficial as this step preferably comprises completing a questionnaire that provides information about Emergency Service and the current PAD status 402 in the organization and/or community. The questionnaire preferably provides a history that details calls concerning cardiac arrests and chest pain that Emergency Services have responded to during a previous period of time. For example, questionnaire preferably requests the history detailing calls concerning cardiac arrests and chest pain, which Emergency Services have responded to during the past three years. An example of a questionnaire is presented in APPENDIX F.

Developing a strategic plan for PAD in the domain 302 also preferably comprises performing a situation analysis 404, which is preferably a current situational analysis, and charts the Emergency Services general knowledge of PAD. An example of an aide to performing this situation analysis is presented as the HeartSave Awareness™ Scorecard in APPENDIX F. However, any number of aides and techniques can be utilized to perform the situational analysis in accordance with the present invention.

In addition, developing the strategic plan for PAD in the domain 302 comprises providing an initial group with an overview of the PAD program 406, and reviewing the questionnaire 408. This initial group subsequently continues with completing a Dangers, Opportunities and Strengths (DOS) worksheet 410, which explores the dangers, opportunities and strengths relating to PAD in their organization. The DOS worksheet provides a diagnostic which is designed to reveal common paralyzing emotions surrounding the initiation of a PAD program in the organization. An example of a DOS worksheet is presented in APPENDIX F.

In addition to completing the questionnaire that provides information about Emergency Services and the current PAD status 402, performing the situational analysis 404, providing an initial group with an overview of the PAD program 406, reviewing the questionnaire with an initial group 408, and completing the DOS worksheet 410, developing the strategic plan for PAD in the domain 302 preferably comprises reviewing the community vision statement for the PAD program 412, preferably with the initial group, to assure applicability and to tailor the program to the needs of the organization and/or community. The community vision statement can be generated with any number of techniques or tools, and an example of such a tool is presented as the “Our HeartSave Awareness™ Goals” worksheet in APPENDIX F.

Developing the strategic PAD plan for the PAD in the domain 302 also preferably comprises identifying obstacles surrounding the implementation of the PAD program in the organization and/or community 414 and discovering solutions to overcome the identified obstacles 416. Identifying obstacles surrounding the implementation of the PAD program 414 and discovering solutions to overcome the identified obstacles 416 can be accomplished with any number of techniques and tools, including the Strategy Circle™ presented in APPENDIX F. A substantial number of solutions to identified obstacles are provided and described in the Tool Box provided in APPENDIX C. Once identifying obstacles surrounding the implementation of the PAD program 414 and discovering solutions to overcome the identified obstacles 416 are completed, developing the strategic plan for the PAD plan 302 preferably continues with generating an action plan 418, preferably a detailed action plan, with the findings from at least these two activities (i.e., from at least step 414 and step 416). The action plan can have any number of formats, can contain data other than the data concerning the identified obstacles and discovered solutions to the obstacles, and can have fewer amounts of data/data categories or a greater amount of data/data categories than all the data obtained with regard to obstacles and obstacle solutions. An example of an action plan is presented in the form of a Project Planner as presented in APPENDIX F.

Developing the strategic plan for the PAD in the domain 302 preferably continues with presenting data from other organizational and/or communal experiences with PAD 420, and identifying potential team members and PAD sites (i.e., HeartSave Awareness™ sites) 422. Identifying potential team members and PAD sites 422 can be accomplished with any number of techniques and based upon any number of factors, such as completing one or more of the “Our HeartSave Awareness™ Corporations” worksheet (Private Corporations, Public Buildings, Municipal Buildings, “Our HeartSave Awareness™ Team” worksheet, and “The HeartSave Awareness™ Referral Form,” as provided in APPENDIX F. This identification process preferably includes identifying individuals, corporations, private, public, and governmental venues as potential members of the HeartSave Awareness™ Team and identifying facilities or areas controlled by these potential team members as PAD sites.

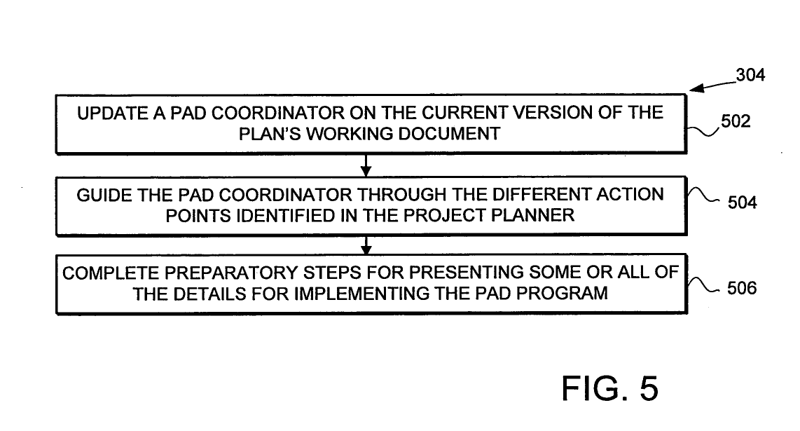

Referring to FIG. 3, after developing the strategic plan for PAD in the domain 302, the method for implementing a PAD program (i.e., a HeartSave Awareness™ program) 300 preferably continues with conducting initial activities for setting up the PAD program in the domain 304. Referring to FIG. 5, some of the initial steps that can be conducted for setting up the PAD program in the domain include, but are not limited to, updating a PAD Coordinator on the current version of the plan's working document 502, guiding the PAD Coordinator through the different action points identified in the Project Planner 504, and completing preparatory steps for presenting some or all of the details for implementing the PAD program 506 to corporations, organizations, entities, individuals that were identified in developing the strategic plan for the PAD in the domain, including, but not limited to, sending out correspondence and invitations to identified PAD sites (i.e., HeartSave Awareness™ sites). Examples of such correspondence and invitations can be found in the TOOL BOX attached as APPENDIX C.

Referring to FIG. 3, the method for implementing the PAD program preferably comprises presenting some or all of the details for implementing the PAD program in the domain 306 after conducting initial activities for setting up the PAD program in the domain 304. Some or all of the details for implementing the PAD program in the domain are preferably presented to corporations, organizations, entities or individuals that were identified in developing the strategic plan for the PAD in the domain 302. This presenting some or all of the details for implementing the PAD in the domain 306 is preferably facilitated by an individual with experience in making the case for PAD. The facilitator preferably employs a presentation which is specifically designed to convince corporations, organizations, entities or individuals to join the PAD program and/or become a PAD site. Examples of such presentations are provided in APPENDIX D and APPENDIX E. In this way, the stage is set for a successful launch of the organization's PAD program by securing in advance a wide variety of corporate PAD sites and participants in advance of the launch.

Presenting some or all of the details for implementing the PAD program in the domain 306 to corporations, organizations, entities or individuals that were identified in the developing the strategic plan in the domain 302 also preferably includes presenting an overview of “Getting Started,” “Communicating Your Message,” and the “Tool Box,” as provided in APPENDIX A, APPENDIX B, and APPENDIX C, respectively. Generally, “Getting Started,” as attached in APPENDIX A, contains information from A-Z about how an Emergency Service implements a community PAD plan in accordance with the methods of the present invention. “Communicating Your Message,” as attached in APPENDIX B, provides information for understanding the media, marketing, and presenting the idea of PAD, and the “Tool Box,” as attached in APPENDIX C, presents the tools to assist in implementing the PAD program and overcoming the obstacles identified by the Strategy Circle™ in the developing the strategic plan (e.g., news releases, PAD support letters, public service announcements for TV and Radio, Automated External Defibrillator fact sheets, PowerPoint® presentations, etc.).

After presenting some or all of the details for implementing the PAD program in the domain 306, the method for implementing the PAD program 300 preferably continues with launching of the PAD program in the domain (i.e., launching HeartSave Awareness™ Program) 308. The corporations, organizations, entities or individuals, which have joined the PAD program as PAD sites, are preferably invited to attend the launching of the PAD program 308, and others, including, but not limited to dignitaries in the community, who will demonstrate their support for the program. Examples of materials and presentations that can be used in the launching of the PAD program in the domain 306 are provided in APPENDIX C. However other techniques and materials can be used in accordance with the present invention.

After launching of the PAD program in the domain 308, selected steps as previously described are preferably repeated at a predefined interval. For example, the following selected steps illustrated in FIG. 4 and FIG. 5 are repeated at a predefined interval including: completing a questionnaire that provides information about Emergency Service and the current PAD status 402; performing a situation analysis 404, which charts the Emergency Services general knowledge of PAD; completing a DOS worksheet 410; which explores the dangers, opportunities and strengths relating to PAD in their organization; reviewing the community vision statement for the PAD program 412; identifying obstacles surrounding the implementation of the PAD program 414; discovering solutions to overcome the identified obstacles 416; presenting data gleaned from other organizations' experiences with PAD; generating an action plan 418; presenting data from other organizational and/or communal experiences with PAD 420; identifying potential team members and PAD sites 422 material to assist with identification of potential team members and PAD sites; updating a PAD coordinator on the current version of the plan's working document 502; guiding the pad coordinator through the different action points identified in the Project Planner 504, completing preparatory steps for presenting some or all of the details for implementing the PAD program 506; presenting some or all of the details for implementing the PAD program 306; and/or launching of the PAD program 308. In accordance with the present invention, any number of intervals can be selected as the predefined intervals. For example, the predefined interval can be a year. The one or more of the selected steps, or subsets of the selected steps as previously described, are preferably repeated at the predefined interval to increase the probability that the current strategic plan details are still relevant and that any new ideas are included in the plan for the next predetermined interval (e.g., the upcoming year). This repetition of steps also provides an opportunity to review the accomplishments and the hurdles of the past year's work in implementing the Public PAD program.

The method for implementing a PAD program 302 preferably comprises proclaiming the domain a PAD organization and/or community 310 (i.e., HeartSave Awareness™ Community). However, this step is preferably conducted only after a determination has been made that the launching of the PAD program in the domain 308 has been successful. If the launching of the PAD program in the domain 308 is unsuccessful, the proclaiming the domain a PAD 310 is preferably not performed in accordance with the present invention. This proclaiming the domain a PAD organization and/or community 310 is preferably conducted at a media event, such as a news conference, with the presence of dignitaries and celebrities. During the media event, an award, such as a plaque or certificate, is preferably presented to the organization and/or community, which honors the achievement in reaching their goal of providing a PAD plan. An example of such a proclamation is provided in the appendices. Once the proclaiming the domain a PAD is conducted or after an unsuccessful launching of the PAD program in the domain 308, the method 300 preferably continues with updating the mapping of domains 312 as successful or unsuccessful implementations of a PAD programs. After the mapping of domains 312, the method 300 preferably continues with identifying a domain for the next phase 301.

From the foregoing description, it should be appreciated that methods for implementing a PAD program are provided in accordance with the present invention that present significant benefits that would be apparent to one or ordinary skill in the art. Furthermore, while a preferred exemplary embodiment has been presented in the foregoing detailed description, which includes the Appendices and references incorporated by reference, it should be appreciated that a vast number of variations in the embodiments exist. Lastly, it should be appreciated that these embodiments are preferred exemplary embodiments only, and are not intended to limit the scope, applicability, or configuration of the invention in any way. Rather, the foregoing detailed description provides those skilled in the art with a convenient road map for implementing a preferred exemplary embodiment of the invention. It being understood that various changes may be made in the function and arrangement of elements described in this detailed description of the drawings without departing from the spirit and scope of the invention.

1. A method for implementing a public access defibrillation (PAD) program, comprising:
identifying a domain for the program;
developing a strategic plan for the program;
conducting initial activities for setting up the program;
presenting details for implementing the program within the domain;
launching the program in the domain;
publicly proclaiming the domain a PAD community or organization if launching is successful; and
mapping the domain as unsuccessful if launch is not successful.

2. The method of claim 1, where developing a strategic plan comprises:
completing a questionnaire regarding emergency services;
providing an initial group with an overview of the program;
reviewing the questionnaire with the initial group;
identifying obstacles surrounding implementation of the program;
identifying solutions to the obstacles;
presenting data from other organizations having a PAD program; and
identifying program team members and PAD sites.

3. The method of claim 1, where conducting initial activities comprises:
updating a PAD coordinator regarding a current version of the program; and
guiding the coordinator through points identified in a project planner.

4. A method for implementing a public access defibrillation (PAD) program, comprising:
activating membership of the program;
analyzing a domain's current condition with respect to PAD;
identifying a PAD team;
identifying a PAD project manager planner;
giving a PAD presentation to an identified organization;
proclaiming launch of the program with a media outlet;
performing an annual review of the program; and
publicly proclaiming successful implementation of the program.

5. A method for implementing a public access defibrillation (PAD) program, comprising:
activating membership of the program;
developing a strategic plan for the program;
identifying a PAD team;
requesting identified organization to install automated external defibrillators;
announcing the program to the public;
executing the strategic plan; and
publicly proclaiming a successful implementation of the program.
